(19) United States
(12) Patent Application Publication
(10) Pub. No.: US 2026/0284232 A1
(43) Pub. Date: Sep. 24, 2026

(54) SYSTEMS AND METHODS FOR IMAGING ANIMALS

(71) Applicant: ARIZONA BOARD OF REGENTS ON BEHALF OF THE UNIVERSITY OF ARIZONA, Tucson,, AZ (US)

(72) Inventors: George SUTPHIN, Tucson, AZ (US); Samuel FREITAS, Tucson, AZ (US); Luis ESPEJO, Tucson, AZ (US)

(21) Appl. No.: 19/472,406

(22) PCT Filed: Apr. 5, 2024
(86) PCT No.: PCT/US2024/023405

§ 371 Date: Oct. 3, 2025

Related U.S. Application Data
(60) Provisional application No. 63/494,334, filed on Apr. 5, 2023.

(51) Int. Cl. A61K 49/00; G06T 7/00; G06T 7/194

(52) U.S. Cl. CPC A61K 49/0097; G06T 7/0016; G06T 7/194; G06T 2207/10056; G06T 2207/10064; G06T 2207/30072

(57) ABSTRACT

Characterizing interactions between hosts and microbes in individual animals includes exposing a population of animals in a first environment to a first microbe for a first period. The first microbe is modified to express a fluorescent protein. Fluorescent images are captured of the individual animals within the population at multiple points in time. The background of the captured fluorescent images is removed. The remaining portions of the images, considered infected portions, are analyzed. Analysis of the infected portions includes identifying the infected area, quantifying the infected area and integrating a fluorescent intensity of the infected area.

### CROSS-REFERENCE TO RELATED APPLICATIONS

This application claims the benefit of U.S. 63/494,334, filed Apr. 5, 2023, which is hereby incorporated by reference as submitted in its entirety.

### STATEMENT REGARDING FEDERALLY SPONSORED RESEARCH OR DEVELOPMENT

This invention was made with government support under Grant No. GM133588 awarded by National Institutes of Health. The government has certain rights in the invention.

### TECHNICAL FIELD

This disclosure is directed to the field of animal imaging, and more particularly to systems and methods for animal imaging for modeling the initiation and progression of microbial colonization in tissues of living animals.

### BACKGROUND

Animals, such as for example Caenorhabditis elegans, may be used to model a variety of biological processes. For example, animals may be used to model the function of the immune system and the response of cells or animals to different diseases. Animals may also be used to model and study the microbiome, such as, for example, the gut microbiome.

The gut microbiome refers to the complex community of microorganisms, including bacteria, viruses, fungi, and archaea, that reside within the gastrointestinal tract, particularly in the colon and intestines. This microbial ecosystem plays a crucial role in maintaining the health and functioning of the digestive system and has far-reaching effects on overall health and well-being.

Studying the gut microbiome has become a rapidly growing field of research, driven by advancements in DNA sequencing technologies and bioinformatics tools. Understanding the complex interactions within the gut microbiome and its impact on health and disease holds significant promise for developing novel therapeutic interventions, such as probiotics, prebiotics, dietary interventions, and fecal microbiota transplantation, aimed at modulating the gut microbiome to promote health and prevent or treat disease.

### SUMMARY

In one aspect, the need for developing an imaging system and analysis pipeline that may employ animals for modeling the initiation and progression of microbial colonization in tissues of living animals by longitudinally monitoring fluorescent localization and intensity of fluorescently labeled microbes. In another aspect, the need for a system that may be used to monitor the co-colonization of multiple microbial strains that express different compatible fluorophores in the tissues of living animals. In yet another aspect, the need for a system that may simultaneously monitor the responsive molecular systems within the same individual animal to microbial colonization using, for example, transgenic animals expressed fluorescent biomarkers that report on molecular processes of interest.

Those needs are met primarily by a process for characterizing interactions between hosts and microbes in individual animals. In a first aspect, the process includes exposing a population of animals in a first environment to a first microbe for a first period sufficient to allow colonization of a tissue, such as the intestine, in a subset of the population of the animals. The microbe is modified to express a fluorescent protein. The process includes moving the animals to a second environment seeded with a second microbe or axenic food source for a second period. The second microbe does not express the fluorescent protein. The second microbe may be alive, or the second microbe may be killed or metabolically inactivated, for example by exposure to heat, ultraviolet radiation, or a chemical, such as paraformaldehyde. The process includes capturing fluorescent images of the individual animals within the population; and analyzing the fluorescent images. In a second aspect, the process includes exposing a population of animals in a first environment to two or more microbes for a first period sufficient to allow gut colonization in a subset of the population of the animals. The microbes are each modified to express a different fluorescent protein with excitation and emission properties that may be independently measured when the microbes are present in the same sample. The process includes moving the animals to a second environment seeded with another microbe for a second period. The final microbe does not express a fluorescent protein. The final microbe may be alive, or the final microbe may be killed or metabolically inactivated, for example by exposure to heat, ultraviolet radiation, or a chemical such as paraformaldehyde. The process may include capturing fluorescent images of the individual animals within the population; and analyzing the fluorescent images. In a third aspect, the process includes exposing a population of animals to one or more microbes. The microbes are each modified to express a different fluorescent protein with excitation and emission properties that may be independently measured. Expression of each fluorescent protein is only activated when the microbe is colonizing a tissue in an animal, for example in response to intestinal pH, the presence of a molecule present in the animal gut, or contact with another microbe in the animal gut, or contact with a protein on a host animal cell, allowing tissue colonizing microbes to be distinguished from microbes in the environment but outside of the host animal. The process includes capturing fluorescent images of the individual animals within the population and analyzing the fluorescent images. Different microbes within each aspect may be of the same species and strain or of different species and strain. Microbes may be bacterial, fungal, or other categories of microbe that colonize animal tissue.

Various additional features and advantages of this invention will become apparent to those of ordinary skill in the art upon review of the following detailed description of the illustrative embodiments taken in conjunction with the accompanying drawings.

### BRIEF DESCRIPTION OF THE DRAWINGS

FIG. 1A shows an example workflow of the disclosed procedure according to one or more embodiments.

FIG. 1B shows an example confocal image of C. elegans following infection with a fluorescently labeled microbe and wash according to one or more embodiments.

FIG. 1C shows an example modified Terasaki tray environment containing individual housing for imaging C. elegans according to one or more embodiments.

FIG. 1D shows example longitudinal tracking of fluorescent infection according to one or more embodiments.

FIG. 1E shows an example heatmap of analysis output of composite data according to one or more embodiments.

FIG. 2A shows an example output of infection area per animal per day in WT animals and the immune pmk-1 mutant according to one or more embodiments.

FIG. 2B shows an example proportion of wild type and pmk-1 mutant animals with detectable infection on the first day of imaging according to one or more embodiments.

FIG. 2C shows an example area of infection in wild type and pmk-1 mutant animals with detectable infection on day 1 after wash step according to one or more embodiments.

FIG. 2D shows an example of the fraction of animals with detectable infections over time according to one or more embodiments.

FIG. 2E shows an example distribution and linear regression of onset of infection again day of death (p<0.05) in wild type animals according to one or more embodiments.

FIG. 2F shows an example distribution and linear regression of onset of infection again day of death (p<0.05) for pmk-1 mutant animals according to one or more embodiments.

FIG. 2G shows an example comparison of time taken from first detection of infection to time of death in wild type and pmk-1 mutant animals according to one or more embodiments.

FIG. 3A shows an example Kaplan-Meier survival curves comparing infected versus non-infected wild type (WT; left) and pmk-1 mutant (right) animals where proportions of the animals lost during challenge are attributed to infected animals on day zero (0) according to one or more embodiments.

FIG. 3B shows an example composite infected and non-infected animals in the wild type (WT) and pmk-1 mutant conditions according to one or more embodiments.

FIG. 3C shows an example comparison of animal resilience to infection in early (days 1 and 2 post infection) and late infections of wild type (WT; left) and pmk-1 mutant (right) animals according to one or more embodiments.

FIG. 3D shows an example of all samples containing detectable infection compared to the time of the animal to its death and a linear regression (dashed line) that is performed to provide a predictive model of infection severity to survival according to one or more embodiments.

FIG. 4A shows an example slope of fluorescent infection area progression calculated in each individual animal in pixels per day in comparison with post survival with linear regression (dotted line) performed for wild type (WT; left, p<0.05) and pmk-1 mutant (right, p<0.05) animals according to one or more embodiments.

FIG. 4B shows an example comparison of infection area progression distribution between wild type (WT) and pmk-1 mutant animals according to one or more embodiments.

FIG. 4C shows an example composite graph of infection area progression including 1) average cumulative sum of infection area of each condition following pathogenic challenge; and 2) weighted for infection prevalence throughout the populations, death during challenge, and death during observation according to one or more embodiments.

FIG. 5A shows example data of infection intensity relative to maximum intensity for all sampled wild-type (WT; left) and immune mutant pmk-1 (right) animals according to one or more embodiments.

FIG. 5B shows example infection intensity of wild type (WT) and pmk-1 mutant animals displaying infection on day one (1) of observation according to one or more embodiments.

FIG. 6A shows an example of infection intensity in each animal in comparison with remaining life with linear regression (dotted line) performed for wild type (WT; left, p<0.05) animals according to one or more embodiments.

FIG. 6B shows an example of infection intensity in each animal in comparison with remaining life with linear regression (dotted line) performed for pmk-1 mutant (WT; left, p<0.05) animals according to one or more embodiments.

FIG. 7A shows a composite heatmaps of GFP labeled E. coli area in wild type (WT; far left) and immune-deficient pmk-1 mutant (left middle) C. elegans, and integrated intensity in wild type (WT; middle right) and immune-deficient pmk-1 mutant (far right) C. elegans according to one or more embodiments.

FIG. 7B shows the fraction of wild type (WT) and pmk-1 mutant animals with detectable infection 1 day after wash according to one or more embodiments.

FIG. 8 shows an example system diagram for an electronic device according to one or more embodiments.

FIGS. 9A-9L show an example graphical user interface (GUI) that may be provided via an electronic display, where the GUIs comprise an image processing GUI than may allow users to manually select image regions that contain artifacts that are removed from analysis according to one or more embodiments.

### DETAILED DESCRIPTION

Animals may be important model systems for host-microbe research. The term microbe as used herein may refer to bacteria, fungi, protists, other parasitic, non-parasitic, commensal, or beneficial microorganisms, combinations thereof, among other possibilities. For example, C. elegans may be an advantageous model system due to the ability of researchers to rapidly quantify the influence of microbial exposure on whole-animal survival and rapidly quantify microbial load in living animals. Some host-microbe interaction studies rely on host group survival and cross-sectional examination of infection severity. Because these studies do not track microbe colonization and survival in the same individuals over time, these studies are unable to connect individual microbial load to physiological or molecular outcomes in that same animal. According to aspects of this invention, imaging systems and methods called Systematic Imaging of Caenorhabditis Killing Organisms (SICKO) is disclosed, which may characterize longitudinal interactions between host and microbes in individual animals (e.g., C. elegans). Specific reference in this disclosure to C. elegans does not limit application of SICKO to these animals. Any number of other animals may be used with SICKO.

The disclosed SICKO system enables researchers to capture dynamic changes in microbial colonization of gut or other tissues between individuals and quantify the impact of bacterial colonization events on host survival. In some embodiments, the SICKO system may demonstrate that colonization of gut or other tissues by microbes (e.g., the bacteria Escherichia coli) may dramatically impact the lifespan of animals (e.g., C. elegans). Additionally, or alternatively, the SICKO system may show that immunodeficient animals, for example C. elegans lacking the pmk-1 gene, cannot significantly alter the progression of bacterial infection, but rather suffer an increased rate of gut colony initiation. The SICKO system may provide a powerful tool for understanding underlying mechanisms of host-microbe interaction and may open a wide avenue for detailed research into therapies that combat pathogen induced illness, the benefits imparted by probiotic microbes, the interplay between multiple microbes and host cells, and understanding the role of the microbiome in host health.

Immune research using mammalian model systems typically rely on in vitro studies in which cell types are isolated and their in vivo environment is simulated, or in animal studies where the initiation and progression of microbial interaction is difficult to quantify. Invertebrate animals may offer the capability to study the immune system in whole animals with short lifespan at an increased throughput. The roundworm C. elegans lacks adaptive and humoral immune components but may recapitulate many genetic and molecular components of innate immunity shared across the animal kingdom. Because C. elegans have a transparent body structure, fluorescently labeled microbes may be easily observed when they colonize individual animals. Under the current standard methodology, C. elegans may be anesthetized for infection visualization. But that may kill the animals, which may limit observation to cross-section studies and may preclude the observation of dynamic changes in infection—timing of infection initiation, growth rate of colonies in host tissues, and the potential reversal of colony growth—in individual animals over time. Little is understood about the relationships between infection dynamics and physiological outcomes, including long term health and survival. Microfluidics devices may enable progression of microbe colonization in individual animals, however, they require the worms to be maintained in a liquid environment, which may cause stress and is not directly analogous to the most common culture environment where C. elegans are maintained on solid nematode growth media (NGM). The SICKO system may enable longitudinal monitoring of microbial colony dynamics in tissues of individual roundworms. Additionally, the SICKO system may be combined with both cultivation techniques. For example, the disclosed SICKO system may be combined with the long-term cultivation of C. elegans that is conducive to longitudinal fluorescent imaging of C. elegans on solid NGM, as an alternative to microfluidics devices. The SICKO system may combine this cultivation technique with systematic image collection and processing of individual C. elegans colonized with fluorescently labeled bacteria to capture the dynamic nature of C. elegans host-microbe interactions across lifespan and the impact on host health and survival. The SICKO may also be used to capture the interplay between microbial colony dynamics and the activity of molecular pathways reported by transgenically expressed fluorescent reporters in the host animal.

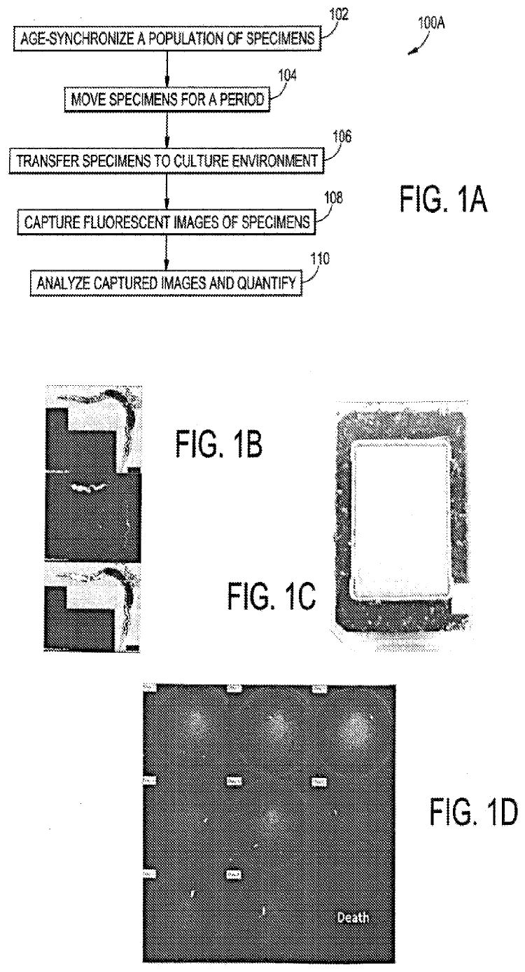

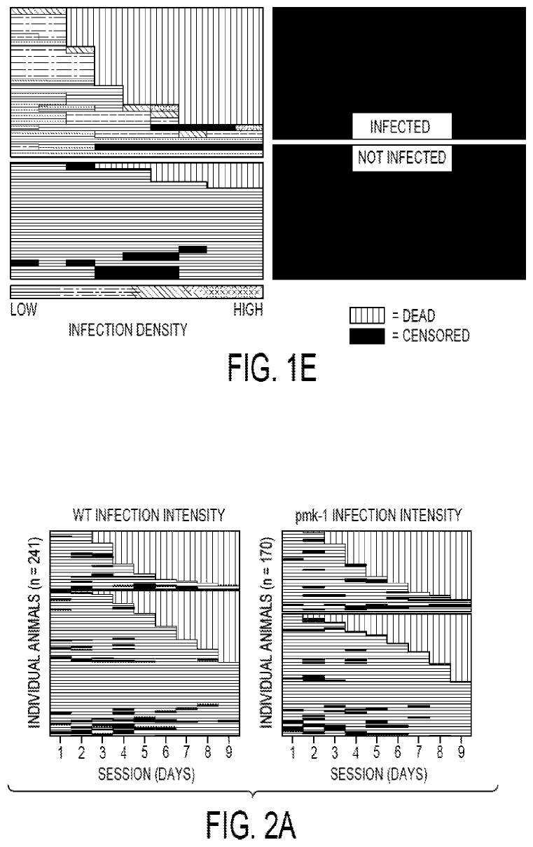

Aspects of this invention are directed to a SICKO process 100A, shown in FIG. 1A. The process 100A includes, for example, at step 102, age-synchronizing a population of C. elegans using standard techniques and initially maintaining animals in group culture on petri plates under standard conditions, for example NGM seeded with E. coli food. The process 100A includes, at step 102, at a user-specified age, exposing the animals to a microbe modified to express a fluorescent protein for a period sufficient to allow gut colonization in a subset of the population. The length of the exposure period may be optimized for each microbial strain of interest (e.g., 7 days starting at day 3 of adulthood for E. coli bacteria strain OP50; 4 days starting at day 3 of adulthood for Pseudomonas aeruginosa bacteria strain PA14). After step 102, the process 100A includes, at step 104, moving the worms to petri plates containing NGM seeded with non-fluorescent microbe for a period (e.g., 16 hours) to allow non-adherent microbes to be passed from the gut and external microbes to be removed from the cuticle via crawling. Step 104 ensures that the only fluorescently labeled microbes remaining are those which have colonized the gut or other tissues. The process 100A includes, at step 106, transferring the worms to a culture environment designed to isolate individual animals on small NGM pads seeded with non-fluorescent microbe. Process 100A includes, at step 108, capturing (e.g., daily) the fluorescent images of each isolated, free-crawling worm and at step 110 analyzing the captured images to quantify the area and intensity of the fluorescent microbe in the gut. In embodiments, the process 100A may include manually or autonomously scoring the worms for survival at regular intervals (e.g., daily).

The SICKO process 100A may generate a rich dataset capturing colonization/infection progression throughout life and lifespan for each animal, such as shown in FIG. 1D. An “infection” may include, for example, a colonization event for a pathogenic microbe. Following microbial exposure and wash, several animals may display established colonization with no detectable extra-corporeal signal, as shown in FIG. 1B. It is recognized that a single-worm culture method may isolate individual C. elegans on small NGM pads, allowing a more direct comparison to previous studies that use C. elegans to examine host-microbe interactions on solid media, and distinguishing SICKO from some systems that use microfluidic systems to monitor microbial infection. The enclosed chamber may be humidified with water absorbing beads, allowing for long-term cultivation of animals for repeated sampling of infection data and monitoring of longevity, as shown in FIG. 1C. The output of SICKO may provide detailed information of infection onset, severity, and progression for every individual animal, in addition to macroscopic insight to population dynamics involving survival and impact of pathogenic challenge or exposure to a beneficial microbe, as shown in FIG. 1D. The infection life history data for a population may be summarized in a heat map that reflects daily infection intensity for each animal, survival, and censoring events, as shown in FIG. 1E. Each row of the heat map may represent an infection state of a single animal. Heatmap areas may capture colonized/infected (above white line) vs. non-colonized/non-infected (below white line) portions of the population. The integrated infection intensity is reflected by the color of the boxes from dark blue (low intensity infection) to yellow (high infection intensity). Red may indicate that the worms is dead. Black may indicate that the data from the animal on that day was censored. Because the animals are imaged while animals are free-crawling, the quantitative longitudinal infection data generated by the SICKO system comes at the cost of identifying the precise location and distribution of the infection within the worm, though images are of sufficient resolution to estimate the tissue in which the colonization event initiated (e.g., pharyngeal pump, vulva, intestine, or anus). The optimized infection and analysis of large populations vastly increases the throughput of this infection system, while capitalization of single animal imaging technology dramatically enhances the biological phenotypes observed regarding host-microbe interaction.

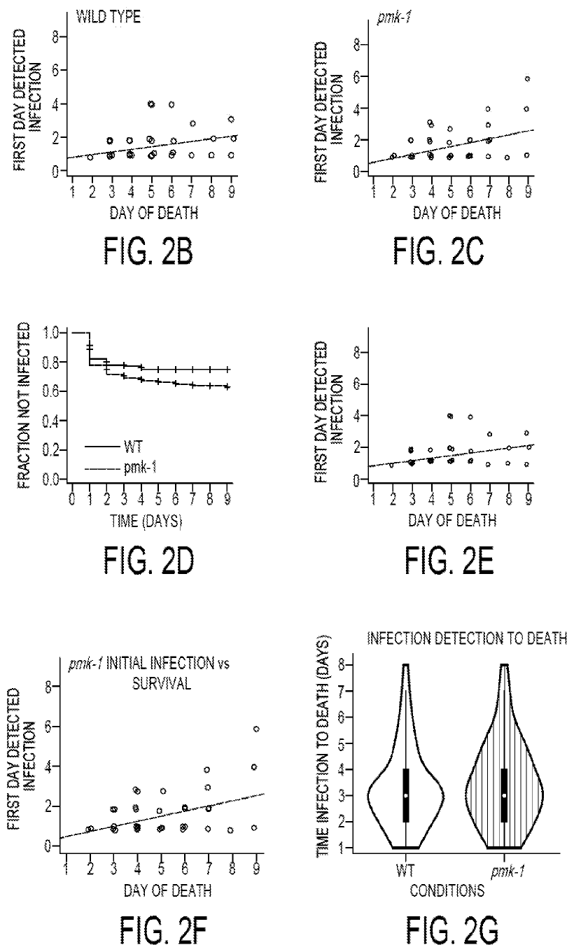

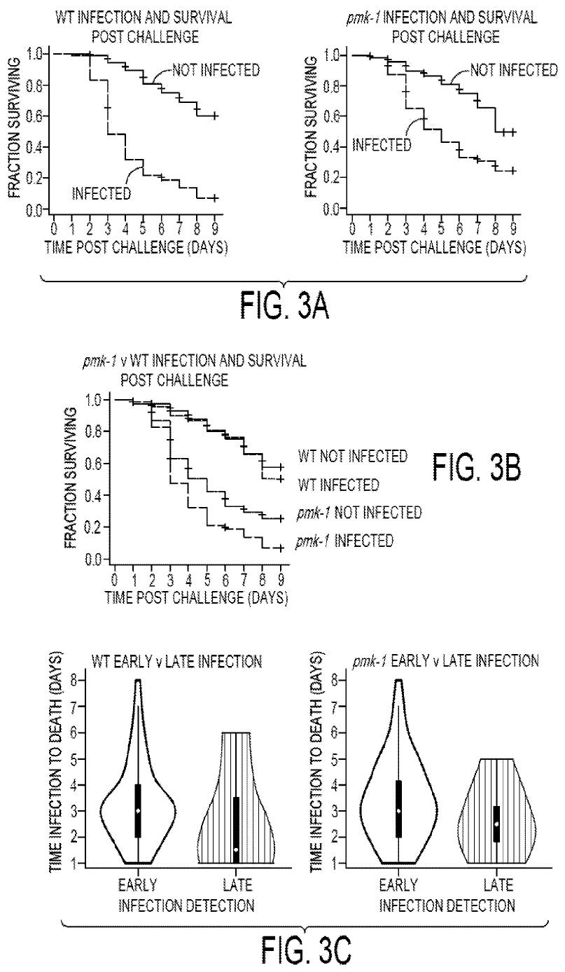

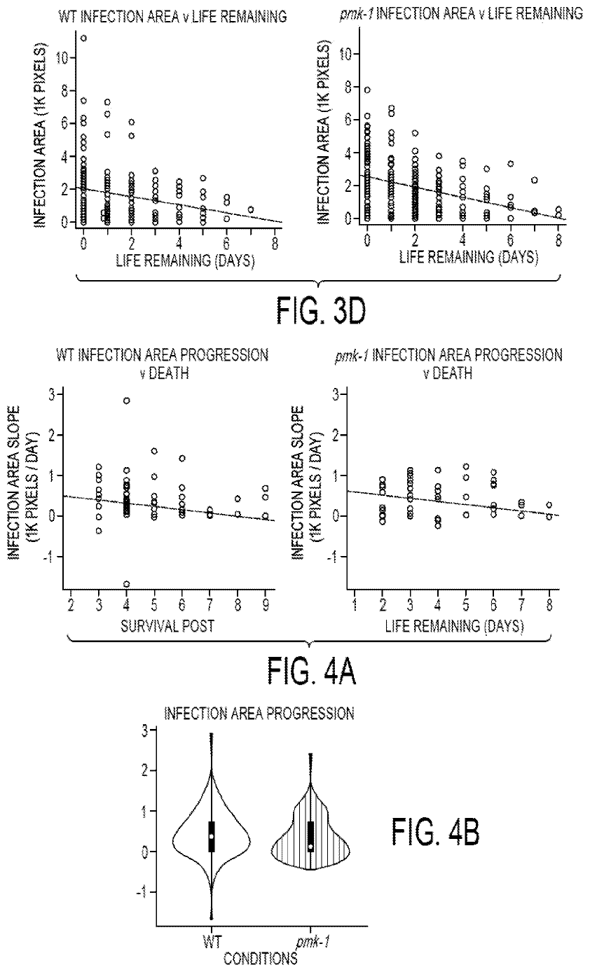

In some embodiments, SICKO may be validated by monitoring a C. elegans strain lacking the p38 MAPK ortholog pmk-1, which has a well-established role in C. elegans immunity, challenged with E. coli strain OP50, as shown in FIGS. 2A-G. OP50 is a strain commonly used as a laboratory food source for C. elegans and is modestly pathogenic to C. elegans. Animals lacking pmk-1 may have reduced lifespan relative to wild type when cultured on OP50 E. coli. Animals may be exposed to OP50 transfected with a plasmid expressing GFP under a ubiquitous trc promoter and monitored for infection. The SICKO system may detect significantly higher rates of infection in the pmk-1 mutant relative to wild type, as shown in FIG. 2A. Additionally, it is recognized that infection occurrence may be significantly correlated with death across both strains individually, as shown in FIG. 3A. To directly compare the SICKO output to the cross-sectional data generated in previous studies, a cross sectional study design may be simulated by examining animals on day one (1), e.g., 16 hours after wash. It is demonstrated that the proportion of each population infected, integrated infection intensity within each animal, and area of infection within each animal were not significantly different for pmk-1 mutants relative to wild type using cross-sectional approach, as shown in FIGS. 2B, 2C, 5B and 5C. The cross-sectional design missed ~29% of infections that were present but below the detection threshold on day one (1) but later expanded to detectable infections. It is determined that SICKO may capture late-emerging infection events through longitudinal imaging, and pmk-1 animals displayed a significantly higher total infection rate, as shown in FIG. 2D. Longitudinal tracking of infection demonstrated that infection onset also significantly impacts time of death following challenge in both the wild-type and pmk-1 mutants, as shown in FIGS. 2E and 2F. But there is no significant difference in the time it takes for an initiated infection to result in death between the two strains, as shown in FIG. 2G. In embodiments, pmk-1 mutant animals do display a higher rate of infection than wild type animals, though this is apparent after allowing infections initially below the detection threshold to progress for several days. SICKO may capture this pattern, while a single examination of cross-section infection rates would not.

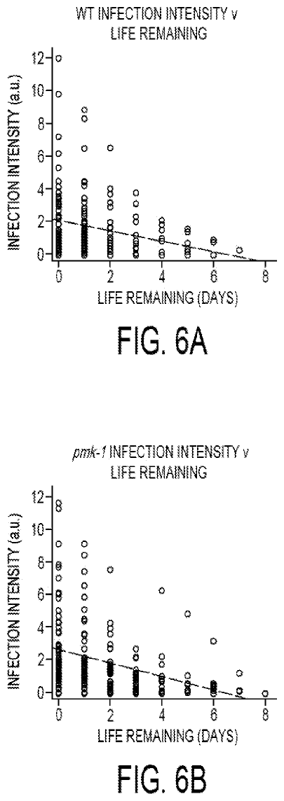

OP50 is a highly utilized C. elegans food sources and its pathogenicity may minimally impact the survival or health of animals with a normal immune system. An advantage of the SICKO system is the ability to differentiate infected vs. non-infected animals within each population. It is determined that the survival of both pmk-1 mutants and wild-type animals was dramatically lower in animals with active OP50 infections relative to animals without detectable infection, as shown in FIG. 3A. pmk-1 mutants were thought to be deficient in several aspects of health even in the absence of a pathogen, contributing to the shortened lifespan. It is determined, using the SICKO system, that wild type and pmk-1 animals without detectable OP50 colonization behave nearly identically in terms of survival, as shown in FIG. 3B. That demonstrates the ability of the SICKO system to characterize biological phenotypes in relationship to the host-pathogen interaction. It is determined that animals with infection onset earlier versus later also appear to have no difference in resilience at the ages in which observation occurs, though there may be a downward trend, as shown in FIG. 3C. It is determined that death typically occurs an average of three (3) days after the onset of detectable infection, as shown in FIG. 2G. The SICKO system allows severity of infection on each day to be isolated and independently compared to remaining survival. It is observed that a direct relationship between infection area remaining survival in both the wild-type and pmk-1 mutants, as shown in FIG. 3D. This survival relationship is similar for integrated infection intensity, as shown in FIGS. 6A and 6B.

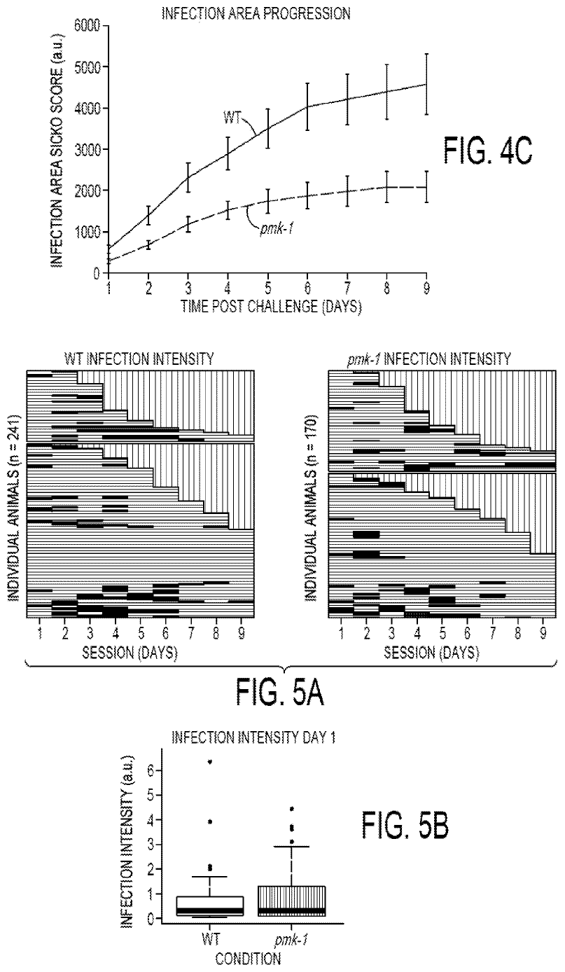

Another advantage of the SICKO system is the capacity to longitudinally track colonization/infection in individual animals. The SICKO system may be used to study the relationship between the rate of progression of colonization/infection within each animal and survival. A linear regression may be performed for infection area versus time in each animal and used the slope as a first-order estimate of colony/infection progression rate within that animal. In both the wild-type and pmk-1 mutants it is determined that OP50 infection progression significantly and negatively correlates with survival among infected individuals, as shown in FIG. 4A. It was unexpectedly determined that OP50 infection area progression was not significantly different between wild-type and immune deficient pmk-1 mutants, as shown in FIG. 4B.

It is determined that the pmk-1 animals were more likely than wild type to die during the wash step and transfer steps introducing a high likelihood for selection bias. It is hypothesized that the pmk-1 animals that died in the wash step were those with early severe infections, and that this may account, at least in part, for the relatively small differences observed in wild type vs. pmk-1 animals, particularly in terms of the survival of animals with detectable infections, as shown in FIG. 3B. No single data type provides a straightforward metric of infection severity in a population at given point in time, in part because, once an animal dies, there is no longer an infection to quantify in terms of area or integrated intensity. To address those issues, a metric has been developed that reflects infection severity, the “SICKO score,” which incorporates information about death prior to observation time (including animals lost during the wash and transfer steps), death during the observation time, and the proportion of the population afflicted by pathogen infection (see below for details). The pmk-1 mutant animals show a consistently higher SICKO score, calculated based on area of infection, throughout their lifespan relative to wild type animals, as shown in FIG. 4C. It is observed that a similar pattern when the SICKO score is derived from integrated infection intensity (data not shown). In summary, the SICKO scores provide a composite metric of immune resilience, tolerance, and survival that better capturing the composite immune deficit of this strain than the independent metrics viewed separately.

The SICKO system may monitor microbial colonization/infection progression over time in isolated individual C. elegans on solid NGM media. Using the immune deficient pmk-1 mutant, the capacity of SICKO is validated to quantify differences in dynamics of bacterial colonization and progression that would be arduous or impossible to reproduce using the dominant cross-sectional approaches. Through longitudinal monitoring of microbial colonization in individual animals, SICKO may help researchers investigate in detail how the presence, severity, and dynamic changes in microbe colonies impact survival. The SICKO system demonstrated that progression and level of infection do not appear significantly different in pmk-1 mutants when compared to wild type, but instead the mutants had an increased capacity to become infected and have a higher capacity for the emergence of previously undetectable infection following first day of observation. Finally, a composite metric of infection severity may be developed within a population, the SICKO score, which incorporates information on prior mortality to adjust the infection severity within the population of interest. Because many components of the SICKO system analysis have been automated, any researcher with moderate experience managing C. elegans and minimal specialized equipment would be capable of utilizing the system and producing rich characterization of microbial colonization phenotypes in a wide range of test conditions. The SICKO system may be combined with emerging automated image collection to enable high-throughput analysis of host-microbe interaction. The SICKO system allows an avenue for detailed testing of novel therapies to combat pathogens, conduct mechanistic studies of innate immune function, and explore the influence of both pathogenic and beneficial bacteria on host health and survival. When combined with transgenic strains expressing fluorescent biomarkers, which are widely available in the C. elegans research community, SICKO enables researchers to directly examine the response of a wide range of molecular processes in host cells to the presence and progression of microbial colonies.

C. elegans Strains and Maintenance

The following provides examples of C. elegans strains and maintenance that may be used with the SICKO system, though other strains and maintenance are possible. C. elegans used in the SICKO system may be cultivated on 60 mm culture plates containing nematode growth media (NGM). NGM plates may be spotted with 300 μl of the E. coli strain OP50 and allowed to dry, for example for 16 hrs. In embodiments, the worms may be allowed a minimum of three passages following recovery from frozen stock or from dauer larvae stage prior to any experiments. The animals may be incubated at 20° C. and may be passed every 3-4 days to fresh NGM plates containing full food. Ages may be synchronized at the egg stage using 10% bleach and 1 mM NaOH solution to eliminate adults, and eggs may be placed on NGM plates spotted with OP50 not containing FUDR. Infections were not witnessed when C. elegans were challenged with fluorescent pathogens during development, and infection may only be seen in moderate numbers if animals were challenged less than five days beyond the L4 stage (data not shown). At the L4 stage animals may be moved to challenge plates or transfer plates containing floxuridine (FUDR) to prevent reproduction until desired age for pathogenic challenge.

### Bacteria Strain Construction

The following is an example bacteria strain construction that may be used with the SICKO system, though other microbial strain constructions may be used. E. coli containing the pMF230 plasmid containing a constitutively active promoter driving expression of eGFP from Addgene and cultivated on 100 uM ampicillin LB agar plates may be used with the SICKO system. Plasmid pFGM1 that contains a gentamycin resistance gene from Addgene and cloned into the pMF230 plasmid creating a constitutively active eGFP plasmid with 10 mg/ml gentamycin resistance may be used. Plasmids may be isolated with a QIAprep Miniprep Kit (Qiagen) and quantified using 260 nm/280 nm absorbance on a Biotek Synergy HI plate reader. OP50 may be made competent and transfected with the GFP plasmid, as previously described. Bacteria colonies displaying green fluorescence may be selected by plating LB supplemented with 10 mg/ml gentamycin.

### Pathogenic Challenge, Wash, and Plating

The following is an example embodiment of pathogenic challenge, wash, and plating that may be implemented with the SICKO system, though other microbial exposures, washes, and plating procedures are possible. The C. elegans may be age synchronized by hypochlorite treatment and plated on to NGM seeded with E. coli strain OP50. At the L4 larval stage, worms may be transferred to NGM plates supplemented with 500 μM FUDR to prevent reproduction and 1 mg/ml gentamycin to select bacteria with the GFP plasmid and spotted with GFP labeled E. coli OP50 bacteria for 7 days (pathogenic challenge). Multiple exposure windows may be examined for wild type C. elegans to GFP-expressing E. coli OP50 to optimize this step. Exposure times may be optimized for different microbes. Wild type animals challenged from L4 until day seven of adulthood results in detectable gut infections in approximately 30% of animals over the subsequent observation period. Exposure for 5 or fewer days starting from the L4 stage results in infection only rarely.

Following challenge, the worms may be washed in M9 buffer supplemented with neomycin (or another antimicrobial compound that the fluorescently labeled microbe is sensitive to) to wash adherent microbes from the outside surfaces of the animals. Worms may then be transferred to fresh NGM plates supplemented with 500 μM FUDR seeded with non-fluorescent E. coli OP50 and placed on the edge of the plate outside of the bacterial spot. This may prompt the worms to crawl toward the food and shed any GFP expressing bacteria attached to their cuticle. The animals may be incubated for at least 16 hours at 20° C. to allow non-adherent GFP-expressing bacteria to fully pass out of their gut. Following this process, the remaining GFP-expressing bacteria may be in adherent colonies in the C. elegans gut. At the end of the 16-hour incubation, randomly selected animals may be transferred to individual wells of single-worm multi-well culture environments, prepared as previously described. Individual agar pads contain non-fluorescent OP50 and gentamycin to prevent colonizing growth during the observation period, and to continue selecting for fluorescent bacteria adherent to the C. elegans gut. The number of animals transferred, the number remaining alive on the plate, and the number that died on the plate following the wash may be recorded for calculation of a SICKO coefficient.

### Imaging and Processing

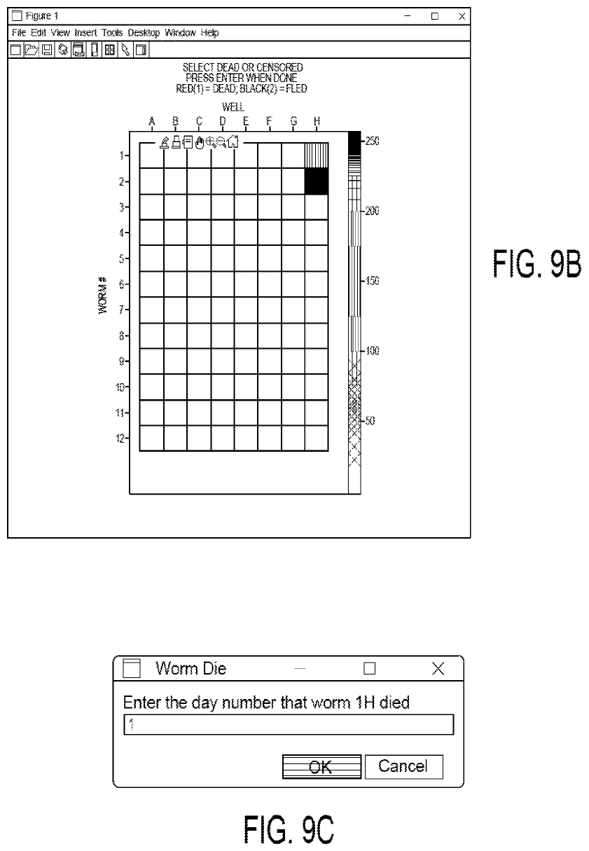

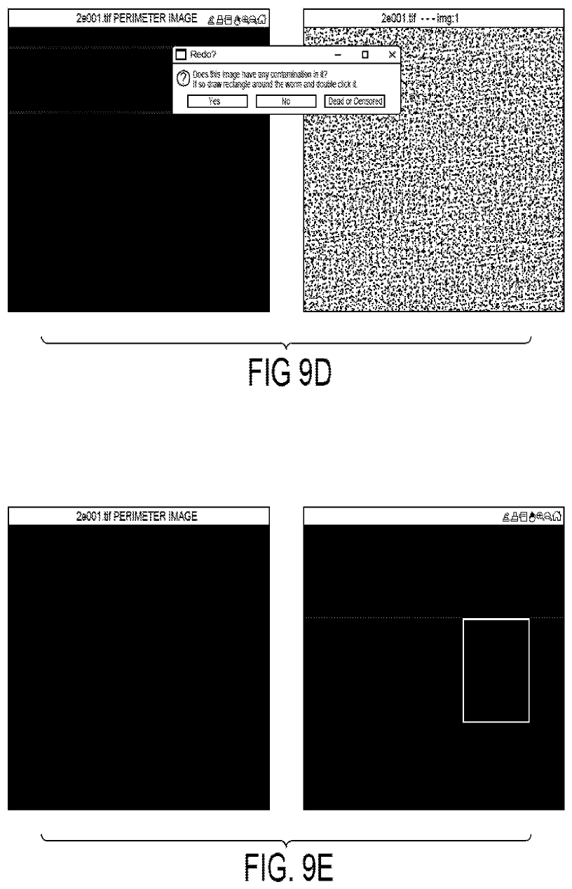

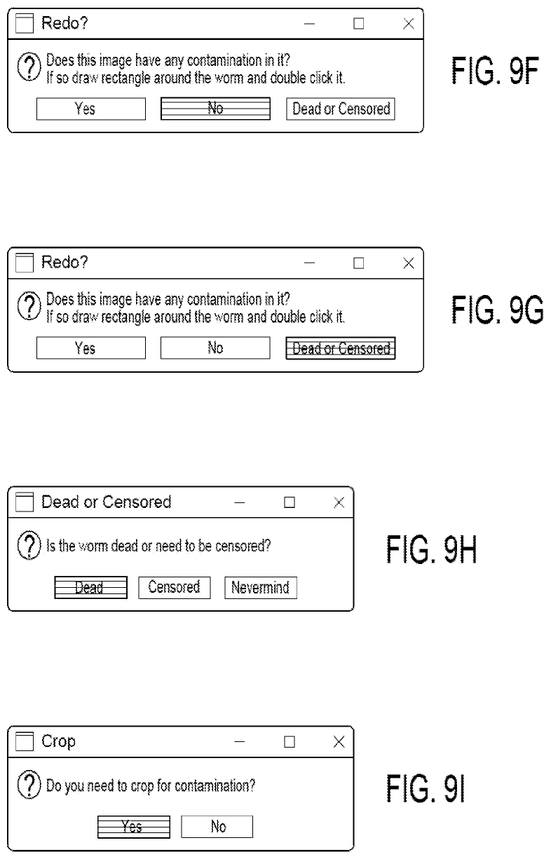

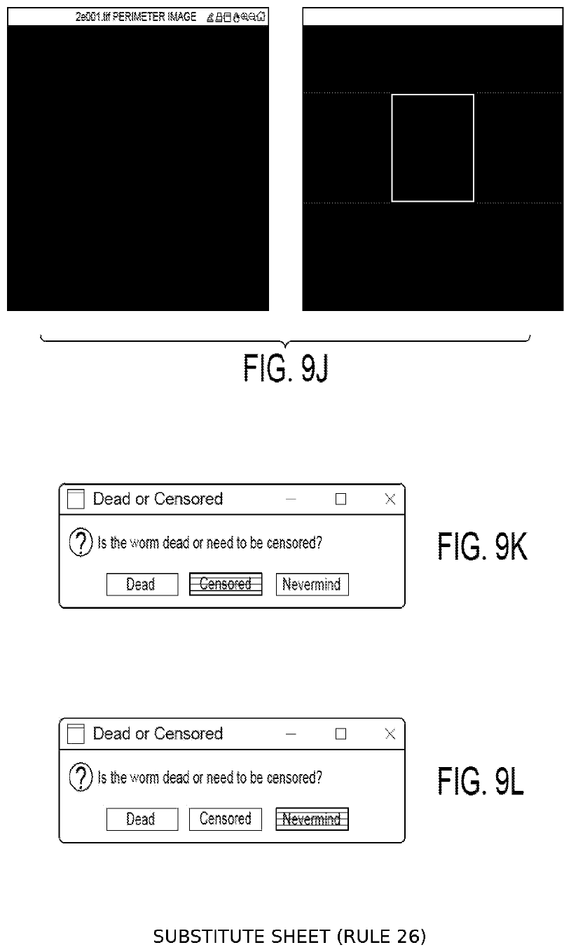

In embodiments, within cascading folders indicating: “Experiment”, “Biological replicate”, “Condition”, and “Day” (denoted with title D #), three repeat images of each animal may be captured and labelled according to the well of the tray. The images may be captured, for example, using 2.5× zoom on a widefield fluorescent microscope as raw TIFs for image analysis. Replicates may ensure the signal is not impacted by worm movement. Animal deaths and fleeing may be recorded following exposure to blue excitation light, which stimulates the animals to move. Image analysis may occur using a graphic user interface (GUI), described herein with respect to FIG. 9. A “SICKO Statistical Testing” script may be executed on the final analyzed csv, where labels may be amended, and the output is throughout graphical and statistical testing of many aspects of dynamic infection in C. elegans.

### Image Analysis and Comprehensive Output

The disclosed technology may include, for example, software (SICKO software) for processing data from the SICKO system. The SICKO software may be provided on a non-transitory computer readable medium (e.g., memory or the like) that when executed by a processor may cause the processor to automatically perform a series of tasks. SICKO software capabilities discussed herein refer to capabilities of a processor executing the SICKO software. The SICKO software may take as input raw fluorescent images each containing a single animal infected with fluorescently labeled microbe, remove background, identify the colony area, and quantify the area and integrated fluorescent intensity of the colony. The SICKO software may display an image processing GUI than may allow users to manually select image regions that contain artifacts (usually fluorescently labeled microbes growing outside of the animal or light pollution) that are removed from analysis. Additional aspects of the SICKO software are described below.

SICKO analysis performed by the SICKO software may utilize a threshold to distinguish colony from background. A user defined threshold may determine a mask that may capture the area in the image representing a microbial colony (the “colony mask” or “infection mask”). To compensate for background differences across the field of view, the SICKO software may perform a background correction radially of the colony mask, which may ensure animal position within the field of view minimally impacts signal intensity and may account for uneven background. The intensity of all pixels within the infection mask may be integrated to calculate the integrated infection intensity for each animal.

Examining the area or integrated intensity of the colony across live animals in a population provides information on the state of the colony at a given point in time, but has limited utility in tracking colony progression over time because it cannot account for animals that died during the microbe exposure (of particular relevance for challenge by pathogenic microbes), during the wash, or during an earlier observation time. Animals that died at earlier stages likely represent a more severe response to microbe exposure, and thus simply quantifying colony area or integrated intensity at a given time point will tend to underestimate the pathogen severity. The SICKO score was developed to provide a quantitative metric of pathogen progress within a population that systematically accounts for the number of animals that died before a given time, including during the post-challenge wash, animals that died at the time of observation, and relative abundance of infection in a population. To determine the SICKO score, a SICKO coefficient is calculated. The SICKO coefficient is used to weight the infection area or integrated infection intensity for each animal. The SICKO coefficient compensates for the loss of individuals to the sampling population from death during challenge and wash (D_{IW}), by using data obtained during the observation phase to make extrapolations. First, the count of animals that died with infection during observation (D_{IO}) is calculated by summing the worms that died during each observation time point (D_{IOi}) across all observation time points as follows:

$${D}_{IO} ( N ) = {\sum }_{i = 1}^{N} {D}_{IOi}$$

D_{IW} is calculated is calculated using number of worms that died in challenge and wash processes (D_{W}), count of animals that died with infection during observation (D_{TIO}), and total number of animals that died during observation (D_{TO}) as follows:

$${D}_{IW} = ( \frac{{D}_{TIO}}{{D}_{TO}} ) {D}_{W}$$

The projected proportion of infected animals from the sampled population (P_{I}) is calculated from the total infected animals observed (T_{IO}), the total number of animals observed (T_{NC}), the total living population after wash (T_{AW}), and D_{IW} as follows:

$${P}_{I} = {D}_{′ W} + ( \frac{{T}_{IO}}{{T}_{NC}} ) {T}_{AW}$$

The SICKO Coefficient (S_{c}) at day N may be determined from P_{I}, T_{AW}, D_{W}, D_{IP}, and D_{IO} in the following equation (i.e., the SICKO equation):

$${S}_{c} ( N ) = ( \frac{1}{\sqrt{1 - \frac{{P}_{i}}{{T}_{AW} + {D}_{W}}}} ) ( \frac{{P}_{I}}{{P}_{I} - ( {D}_{IW} + {D}_{TO} ( N ) )} )$$

The SICKO Coefficient may compensate for animal death and differences in pathogenicity. Increased infected population and death associated with infected animals both positively impacts the SICKO score. The first half of the SICKO equation weighs healthy population, or projected population not impacted by infections. The second half of the SICKO equation is tabulated daily, increasing due to accumulating deaths associated with infection. The SICKO score is applied to the cumulative sum of either infection area (as shown in Section c of FIG. 4) or intensity, for each individual animal to not reduce infection growth due to zeros from animal death. The average SICKO score and standard area may then be computed for animals within each test condition by pairwise permutation testing. In summary, the SICKO score weighs colonization prevalence within the population and mortality associated with infection to provide a comprehensive measure of pathogenicity due to host-microbial interaction.

The following describes aspects of the figures. FIG. 1A shows a workflow of the SICKO procedure. Host animals are harvested and allowed to grow to adulthood. Adult worms are challenged with fluorescent pathogen. Following challenge, worms are washed and then allowed to crawl in non-fluorescent live bacteria, non-fluorescence killed or metabolically inactivated bacteria, or axenic food for 24 hours to remove non-corporeal bacteria. Worms are placed in solo housing and imaged daily. Images are analyzed and compiled. FIG. 1B shows a confocal image of host animals following colonization and wash. FIG. 1C shows a modified Terasaki tray containing individual housing for imaging C. elegans. FIG. 1D shows longitudinal tracking of fluorescent colonization by GFP labeled E. coli in a representative host C. elegans. FIG. 1E shows a heatmap of SICKO analysis output of composite data.

FIG. 2A shows a composite SICKO heatmap of GFP labeled E. coli area in wild type and immune-deficient pmk-1 mutant C. elegans. FIG. 2B shows the proportion of animals with detectable infection on the first day of imaging. FIG. 2C shows an area of infection in animals with detectable infection on day 1 after wash step. FIG. 2D shows the Fraction of WT and pmk-1 animals with infection over time. FIG. 2E shows a distribution and linear regression of WT onset of infection again day of death. (p<0.05). FIG. 2F shows a distribution and linear regression of immune mutant onset of infection again day of death. (p<0.05). FIG. 2G shows a comparison of time taken from first detection of infection to time of death.

FIG. 3A shows survival of WT animals comparing infected versus non-infected C. elegans (left p<0.05) and pmk-1 mutants (right<0.05). FIG. 3B shows composite survival of infected and non-infected animals in the WT and pmk-1 mutant conditions. FIG. 3C shows a comparison of animal resilience to infection in early (day 1 and 2 post infection) and late infections of WT animals (left<0.05) and pmk-1 mutants (right<0.05). FIG. 3D shows that for all samples containing detectable infection, infection area was compared in all samples containing detectable infection were compared to the time remaining between the time of observation and the animal's death and a linear regression (red dotted line) is performed to provide a predictive model of infection severity to survival.

FIG. 4A shows the slope of fluorescent infection area progression in pixels per day in comparison with post survival challenge with linear regression (red dotted line) performed for WT animals (left, p<0.05) and pmk-1 mutants (right, p<0.05). FIG. 4B shows a comparison of infection area progression distribution between WT and pmk-1 mutants. FIG. 4C shows a SICKO composite graph of infection area progression. Average cumulative sum of infection area of each condition following pathogenic challenge. Weighted for infection prevalence throughout the populations, death during challenge, and death during observation.

FIG. 5A shows a composite SICKO heatmap of GFP labeled E. coli integrated intensity in wild type (left) and immune-deficient pmk-1 mutant (right) C. elegans. FIG. 5B shows infection intensity of animals displaying infection on day 1 of observation.

FIG. 6 shows that for all samples containing detectable infection, infection intensity was compared in all samples containing detectable infection were compared to the time remaining between the time of observation and the animal's death and a linear regression (red dotted line) is performed to provide a predictive model of infection severity to survival.

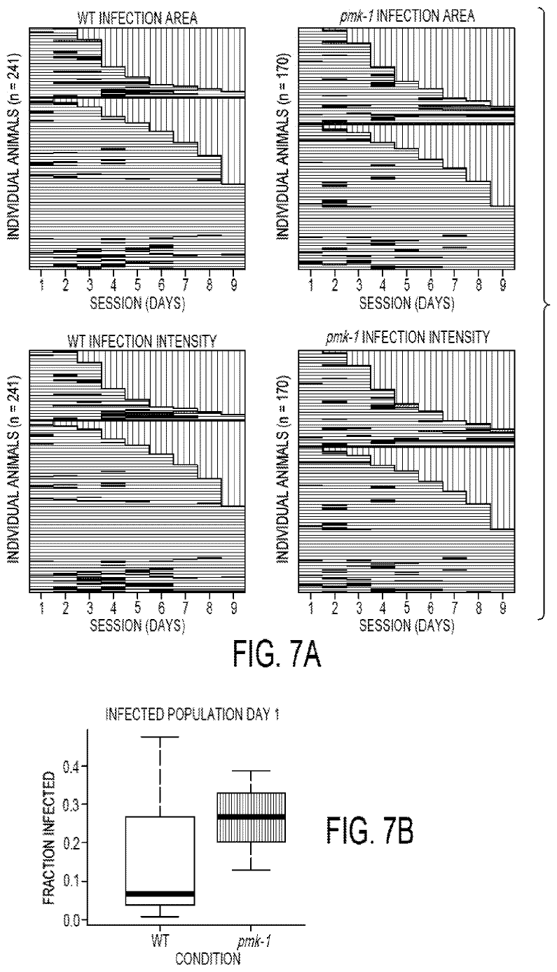

FIG. 7A shows a composite SICKO heatmap of GFP labeled E. coli area in wild type (far left) and immune-deficient pmk-1 mutant (left middle) C. elegans, and integrated intensity in wild type (middle right) and immune-deficient pmk-1 [italics] mutant (far right) C. elegans. FIG. 7B shows the fraction of animals with detectable infection one (1) day after wash.

### Example Multifunction Device

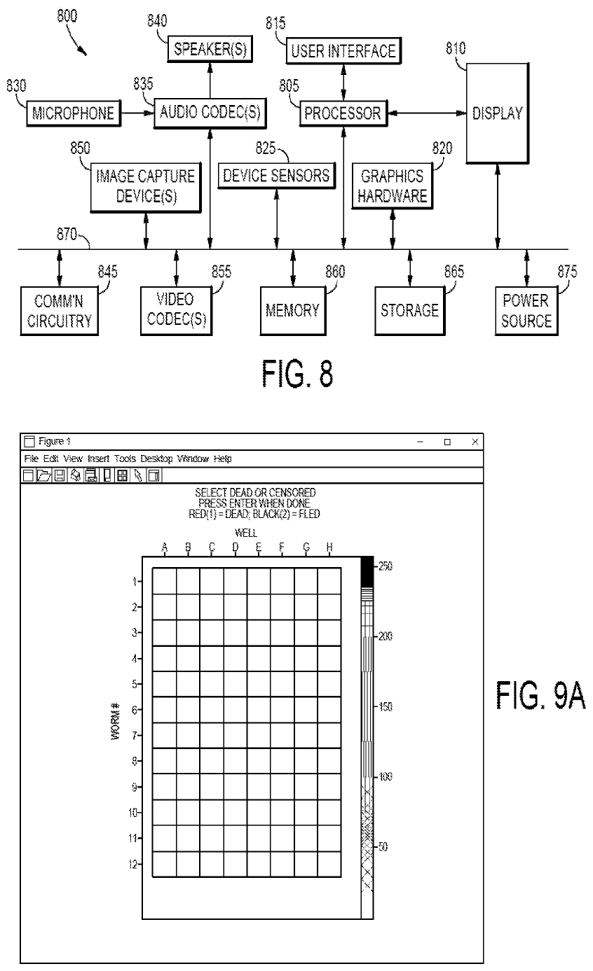

Referring now to FIG. 8, a simplified functional block diagram of illustrative multifunction device 800 is shown according to one embodiment. Multifunction electronic device 800 may include processor 805, display 810, user interface 815, graphics hardware 820, device sensors 825 (e.g., proximity sensor/ambient light sensor, accelerometer and/or gyroscope), microphone 830, audio codec(s) 835, speaker(s) 840, communications circuitry 845, digital image capture circuitry 850, video codec(s) 855 (e.g., in support of digital image capture unit), memory 860, storage device 865, power source 875, and communications bus 870. Multifunction electronic device 800 may be, for example, a digital camera or a personal electronic device such as a personal digital assistant (PDA), personal music player, mobile telephone, or a tablet computer.

Processor 805 may execute instructions necessary to carry out or control the operation of many functions performed by device 800 (e.g., such as the capture and/or processing of image data as disclosed herein). Processor 805 may, for instance, drive display 810 and receive user input from user interface 815. User interface 815 may allow a user to interact with device 800. For example, user interface 815 may take a variety of forms, such as a button, keypad, dial, a click wheel, keyboard, display screen and/or a touch screen. Processor 805 may also, for example, be a system-on-chip such as those found in mobile devices and include a dedicated graphics processing unit (GPU). Processor 805 may be based on reduced instruction-set computer (RISC) or complex instruction-set computer (CISC) architectures or any other suitable architecture and may include one or more processing cores. Graphics hardware 820 may be special purpose computational hardware for processing graphics and/or assisting processor 805 to process graphics information. In one embodiment, graphics hardware 820 may include a programmable GPU.

Microphone 830 may capture audio data, such as digital audio data. Output from microphone 830 may be processed, at least in part, by audio codec(s) 830 and/or processor 805, and/or a dedicated audio processing unit (not shown). Audio data that is captured may be stored in memory 860 and/or storage 865. Image capture circuitry 850 may capture still and/or video images. Output from image capture circuitry 850 may be processed, at least in part, by video codec(s) 855 and/or processor 805 and/or graphics hardware 820, and/or a dedicated image processing unit (not shown). Images so captured may be stored in memory 860 and/or storage 865.

Sensor and camera circuitry 850 may capture still and video images that may be processed in accordance with this disclosure, at least in part, by video codec(s) 855 and/or processor 805 and/or graphics hardware 820, and/or a dedicated image processing unit incorporated within circuitry 850. Images so captured may be stored in memory 860 and/or storage 865. Memory 860 may include one or more different types of media used by processor 805 and graphics hardware 820 to perform device functions. For example, memory 860 may include memory cache, read-only memory (ROM), and/or random-access memory (RAM). Storage 865 may store media (e.g., audio, image and video files), computer program instructions or software, preference information, device profile information, and any other suitable data. Storage 865 may include one more non-transitory computer-readable storage mediums including, for example, magnetic disks (fixed, floppy, and removable) and tape, optical media such as CD-ROMs and digital video disks (DVDs), and semiconductor memory devices such as Electrically Programmable Read-Only Memory (EPROM), and Electrically Erasable Programmable Read-Only Memory (EEPROM). Memory 860 and storage 865 may be used to tangibly retain computer program instructions or code organized into one or more modules and written in any desired computer programming language. When executed by, for example, processor 805 such computer program code may implement one or more of the methods described herein.

### Example GUIs

FIGS. 9A-9L show an example graphical user interface (GUI) that may be provided via an electronic display, where the GUIs comprise an image processing GUI that may allow users to manually select image regions that contain artifacts that are removed from analysis according to one or more implementations of the disclosed technology. Referring to FIGS. 9A and 9B, a GUI may be provided via a display screen. The GUI enables a computer user to select dead or censored image regions that contain artifacts that are to be removed from analysis. Referring to FIG. 9C, the computer user may be prompted to indicate the day number on which a certain specimen died or fled. Referring to FIG. 9D, the GUI may provide a screen prompting the computer user to indicate whether any image regions include any contamination. If so, the computer user may draw a rectangle around a specimen, as illustrated in FIG. 9E. In some embodiments, the computer user may be prompted to identify contaminations in a series of images (FIGS. 9F-9I). To keep a portion of an image, the computer user may draw a rectangle around the portion of the image that is to be kept (FIG. 9J). If a specimen is dead or needs to be censored, the computer user may indicate as such by selecting the appropriate button in FIG. 9K. Otherwise, referring to FIG. 9L, the computer user may select the button ‘Nevermind’ to move on to the next image for processing. Additional embodiments may be implemented without departing from the spirit and scope of the disclosed technology. The GUI screens described herein are merely provided as illustrative examples and are not meant to be limiting.

### ADDITIONAL CONSIDERATIONS

It will be appreciated that the foregoing description provides examples of the invention. However, it is contemplated that other implementations of the invention may differ in detail from the foregoing examples. All references to the invention or examples thereof are intended to reference the particular example being discussed at that point and are not intended to imply any limitation as to the scope of the invention more generally. All language of distinction and disparagement with respect to certain features is intended to indicate a lack of preference for those features, but not to exclude such from the scope of the invention entirely unless otherwise indicated.

1. A method, comprising:
exposing a population of animals in a first environment to a first microbe for a first period, wherein the first microbe is modified to express a fluorescent protein;
capturing, after exposing the population of animals to the first microbe, fluorescent images of the individual animals within the population at multiple points in time; and
analyzing the fluorescent images.

2. The method of claim 1, wherein the first environment includes a plurality of microbes, the plurality of microbes including the first microbes each of the plurality of microbes modified to express a different fluorescent protein with measurable excitation and emission properties.

3. The method of claim 2, wherein expression of each fluorescent protein is only activated when the microbe is colonizing a tissue in an animal.

4. The method of claim 3, wherein expression of each fluorescent protein is only activated when the microbe is colonizing a tissue in an animal in response to intestinal pH or contact with a protein on a host animal cell.

5. The method of claim 2, wherein the plurality of microbes are the species and strain, different species and strains, or combinations thereof.

6. The method of claim 1, further comprising moving the animals to a second environment, before capturing the fluorescent images, wherein the second environment is seeded with a second microbe for a second period, and wherein the second microbe does not express the fluorescent protein.

7. The method of claim 1, wherein the animals are Caenorhabditis elegans.

8. The method of claim 1, wherein the first period of time is sufficient to allow gut colonization in a subset of the population of the animals.

9. The method of claim 1, wherein the microbe is a bacteria.

10. The method of claim 1, wherein the microbe is a fungus.

11. The method of claim 1, wherein the microbe is another parasitic organism.

12. The method of claim 1, wherein analyzing the fluorescent images comprises quantifying the area and intensity of the fluorescent microbe consumed by the animals.

13. The method of claim 1, wherein analyzing the fluorescent images comprises removing the background of the fluorescent images.

14. The method of claim 1, wherein analyzing the fluorescent images comprises analyzing an infected area of the individual animals.

15. The method of claim 14, wherein analyzing the infected area comprises at least one of identifying the infected area, quantifying the infected area, or integrating a fluorescent intensity of the infected area.

16. The method of claim 14, wherein analyzing the infected area comprises utilizing a threshold to distinguish the infected area from background.

17. A non-transitory computer-readable medium comprising instructions that, when executed by a processor, implement:
exposing a population of animals in a first environment to a first microbe for a first period, wherein the first microbe is modified to express a fluorescent protein;
capturing, after exposing the population of animals to the first microbe, fluorescent images of the individual animals within the population at multiple points in time; and
analyzing the fluorescent images.

18. The non-transitory computer-readable medium of claim 17, wherein the first environment includes a plurality of microbes, the plurality of microbes including the first microbes each of the plurality of microbes modified to express a different fluorescent protein with measurable excitation and emission properties.

19. The non-transitory computer-readable medium of claim 18, wherein expression of each fluorescent protein is only activated when the microbe is colonizing a tissue in an animal.

20. The non-transitory computer-readable medium of claim 19, wherein expression of each fluorescent protein is only activated when the microbe is colonizing a tissue in an animal in response to intestinal pH or contact with a protein on a host animal cell.

21.-48. (canceled)
